(19) United States
(12) Patent Application Publication
(10) Pub. No.: US 2013/0165781 A1
(43) Pub. Date: Jun. 27, 2013

(54) INTEGRATED DISPLAY OF ULTRASOUND IMAGES AND ECG DATA

(71) Applicants: Michael Daniel Cardinale, Nottingham, NH (US); Ivan Salgo, Pelham, MA (US)

(72) Inventors: Michael Daniel Cardinale, Nottingham, NH (US); Ivan Salgo, Pelham, MA (US)

(73) Assignee: KONINKLIJKE PHILIPS ELECTRONICS N.V., EINDHOVEN (NL)

(21) Appl. No.: 13/638,755

(22) PCT Filed: Mar. 23, 2011
(86) PCT No.: PCT/IB2011/051236

§ 371 Date: Jan. 29, 2013

Related U.S. Application Data
(60) Provisional application No. 61/320,205, filed on Apr. 1, 2010.

(51) Int. Cl. A61B 8/00; A61B 8/08; A61B 5/0472; A61B 5/04; A61B 5/0432; A61B 5/044; A61B 8/14

(52) U.S. Cl. CPC A61B 8/463; A61B 5/044; A61B 8/0883; A61B 8/5284; A61B 8/145; A61B 8/4444; A61B 8/4488; A61B 8/5207; A61B 8/466; A61B 5/04012; A61B 5/0432; A61B 5/0472
USPC 600/440

(57) ABSTRACT

A display system for ultrasound images and ECG data produces a common display of a cardiac ultrasound image of a given view and ECG traces relevant to that ultrasound view. The ECG traces relate to the heart anatomy seen in the ultrasound image. The user is given the ability to select certain ECG lead signals for display in conjunction with specific views of the heart. ST elevation values for the ECG leads may also be shown to enable the clinician to correlate electrical abnormalities with anatomical abnormalities of the ultrasound image such as abnormal wall motion or thickening. The ST elevation values are displayed on a bullseye chart in association with heart regions related to the leads for which the ST values were detected.

This invention relates to medical diagnostic systems and, in particular, to diagnostic systems which display both ultrasound images and ECG lead data for cardiac assessment.

An established diagnostic exam of cardiac performance is the stress exam. Two kinds of stress exams are commonly performed. One is the stress echocardiogram study, in which the heart is imaged ultrasonically. In a stress echo exam, ultrasound images of the heart are acquired at the outset when the patient is resting. These images are standard cross-sectional images of long axis and short axis views of the heart during the rest stage. The patient then exercises to raise the heart rate above a given level. This may be done by having the patient run on a treadmill, and it may also be done by injection of a pharmacological agent. The same standard images are acquired during the exercise stage when the heart is beating at a high rate. The pre- and post-exercise images are then compared, generally by first synchronizing the different heartbeats of the image loops so that they move together. Characteristics which are assessed include wall motion and systolic thickening (tissue deformation) of the myocardium. Qualitative and quantitative analysis is performed on the images using tissue Doppler, speckle image analysis, i.e., strain quantification analysis, or any other ultrasonic detection of myocardial deformation. Left ventricular filling, ejection fraction, and ejection velocities may also be assessed.

In ECG stress, the ECG lead signals are likewise recorded during both resting and exercise stages. The ECG lead signals are analyzed for ST-elevation indicative of myocardial infarction. Generally this is a 12-lead exam. The ECG signals acquired by an ultrasound system during the ultrasound exam use only three electrodes for the right arm, left arm, and the left leg. This is because the ultrasound ECG leads are only needed to acquire an R-wave for heartbeat gating. The three leads are insufficient to acquire more subtle waveform characteristics such as the P-wave and the T-wave.

In many cases the stress echo and stress ECG studies are combined. The clinician will then review the information gathered by both techniques, looking for electrical changes and differences in the ECG data and motion and anatomical changes and differences in the ultrasound images. The clinician will often do this by looking at ultrasound images on a monitor while balancing the stripcharts with the ECG data on the clinician's lap and glancing from one to the other to make comparisons and look for correlations.

It would be desirable to provide a way for the clinician to view the results of both studies at the same time on the same display. It would further be desirable to display an ultrasound image or image loop simultaneously with the ECG lead traces that relate most closely to the particular ultrasound view of the heart being observed. It would also be desirable to enable the clinician to select specific lead traces for simultaneous viewing with a particular ultrasound view.

In accordance with the principles of the present invention, a diagnostic ultrasound and ECG display system simultaneously presents both ultrasound images and ECG lead traces on the same display. The display is configured to show an ultrasound image or image loop of a particular view together with the ECG lead traces that are most relevant to the ultrasound view of the heart being observed. In a constructed embodiment the clinician is able to select specific ECG traces for simultaneous display with the ultrasound image that the clinician believes are most relevant to the ultrasound view being displayed.

In the drawings:

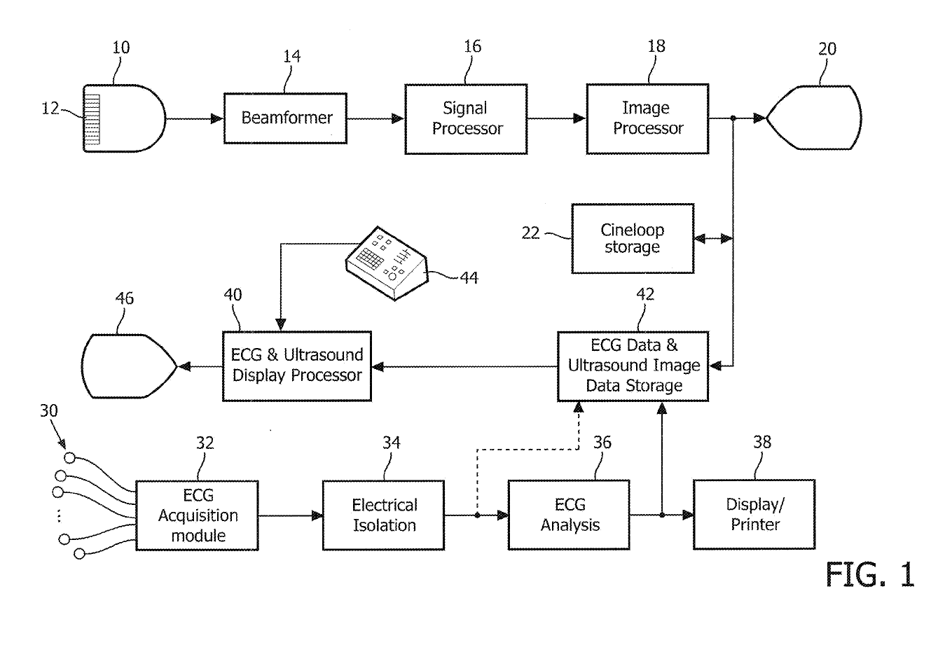

FIG. 1 illustrates in block diagram form an ultrasound and ECG diagnostic system constructed in accordance with the principles of the present invention.

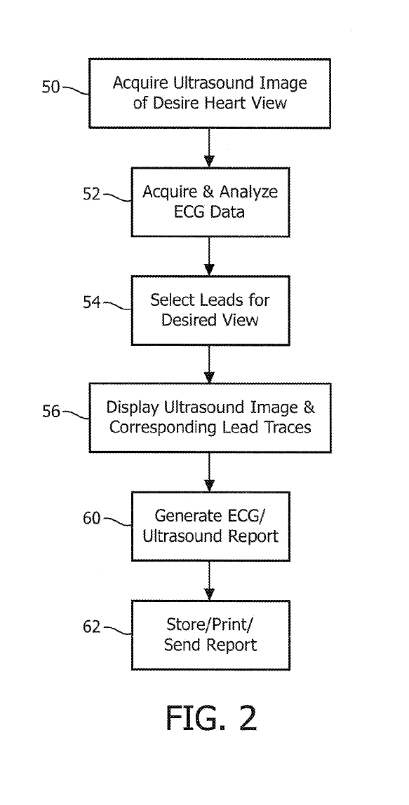

FIG. 2 illustrates a flowchart for acquiring and displaying selected ultrasound images and ECG lead traces in accordance with the present invention.

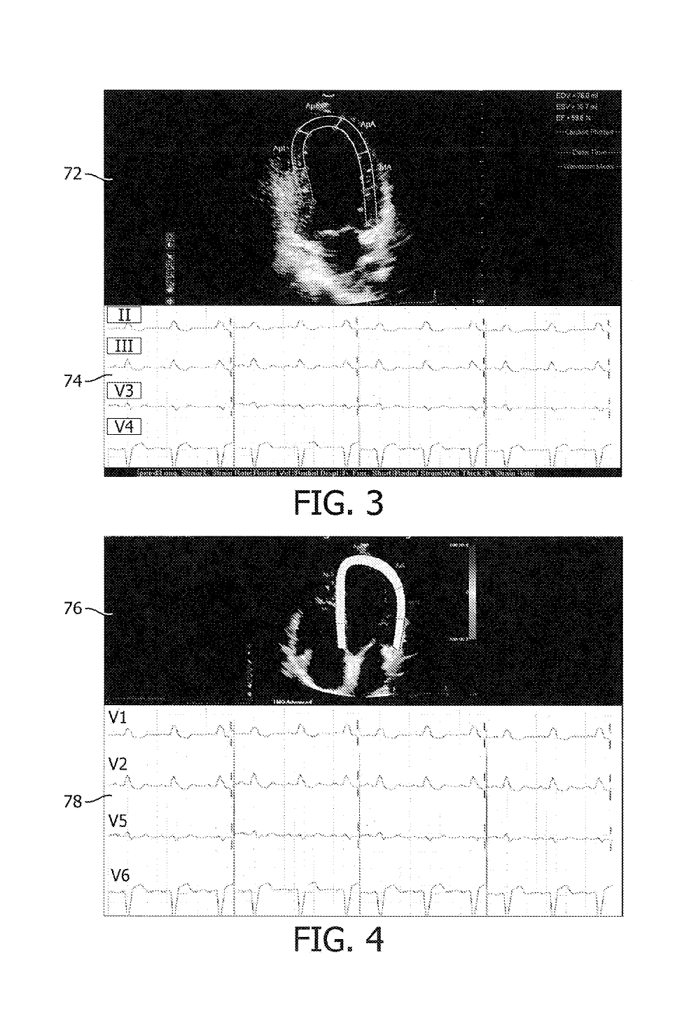

FIGS. 3 and 4 illustrate apical ultrasound views of the heart and associated ECG lead traces on the same display.

FIG. 5 illustrates a short axis ultrasound view of the heart and associated ECG lead traces on the same display.

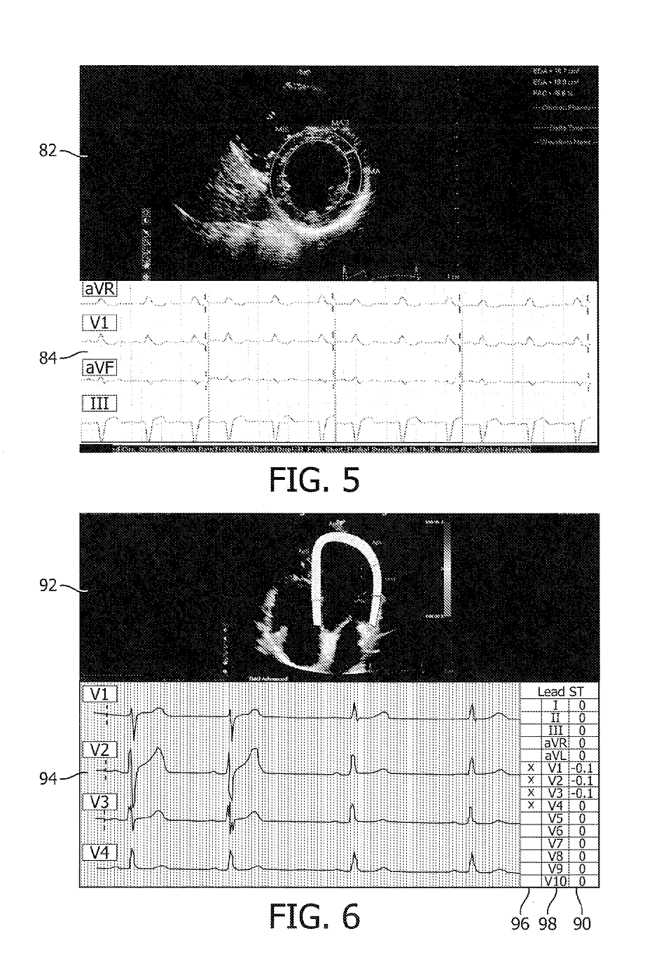

FIG. 6 illustrates a combined ultrasound image display and ECG trace display with user selection of the specific lead traces to be shown for the particular ultrasound image view.

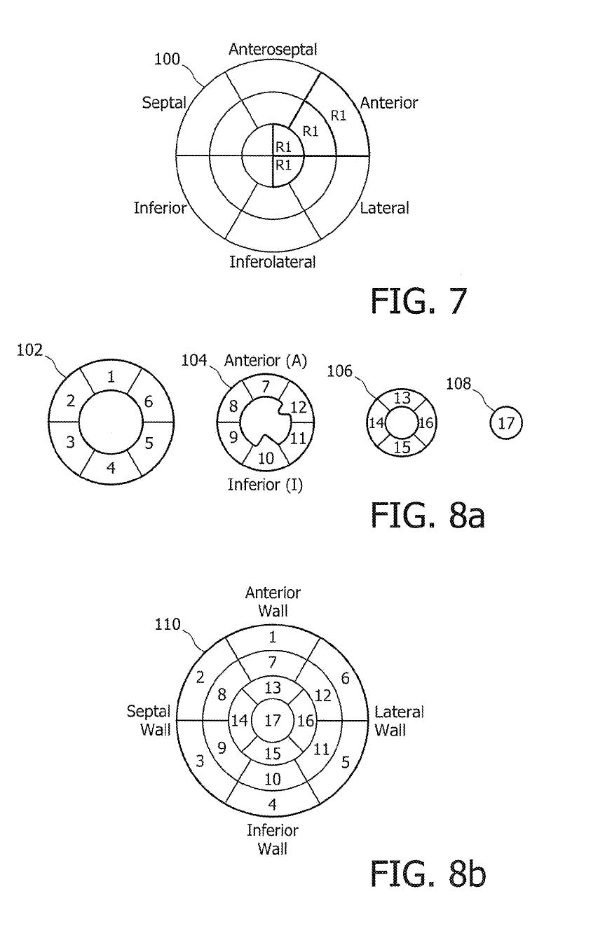

FIG. 7 illustrates a bullet scorecard which is visually marked to indicate suspect regions of a heart.

FIGS. 8a and 8b illustrate the layout of an ECG bullseye chart.

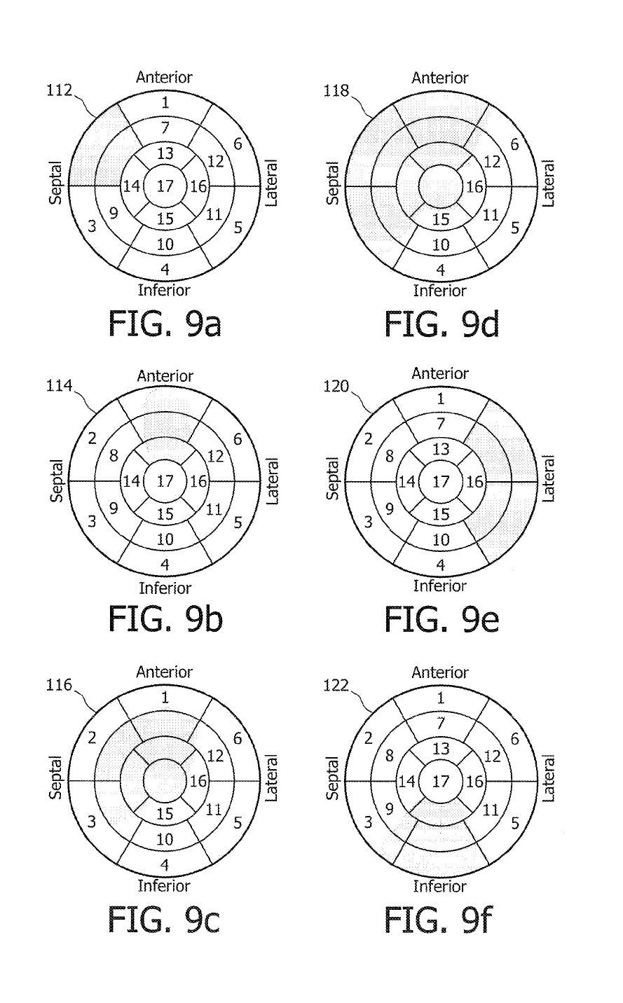

FIGS. 9a-9f illustrate 3D bullseye charts which have been annotated with ST-elevation data to indicate regions of the heart which may have experienced infarction.

Referring first to FIG. 1, a display system for ultrasound images and ECG lead traces is shown in block diagram form. The major subsystems of an ultrasound system are shown at the top of the drawing. An ultrasound probe 10 with an array transducer 12 transmits ultrasound waves to the heart of a patient and receives echoes in response. The echo signals received by the individual transducer elements of the array are processed by a beamformer 14 to form coherent echo signals relating to specific points in the body. The echo signals are processed by a signal processor 16. Signal processing may include separation of harmonic echo signal components for harmonic imaging and clutter removal, for example. The processed signals are arranged into images of a desired format by an image processor 18. The images are displayed on an ultrasound system display 20. Live image loops are stored in Cineloop® storage 22 for later recall an analysis.

The ultrasound images used in stress echo are real time (live) images of the heart as it is beating. A nominal display rate for live ultrasound images is 30 frames per second. The images may be either two-dimensional or three-dimensional images of the heart. In the examples shown below, two-dimensional images are shown. The standard views for stress echo studies are parasternal long axis views such as the parasternal 3-chamber view, and parasternal short axis views at the base, mid-cavity, and apical levels of the heart. Parasternal images are acquired by transmitting and receiving ultrasound signals through the intercostal regions between the ribs. Other standard views in stress echo exams include apical 4-chamber, 2-chamber and long axis views. Apical views are acquired by placing the probe below the rib cage and transmitting and receiving ultrasound while the probe is viewing the heart from below, from the apex. The outflow tract of the heart is visible in the 3-chamber view, whereas the outflow tract cannot be seen in a 4-chamber view. A 2-chamber view shows only the left ventricle and the left atrium. The most common short axis view used is the mid-view, which captures the papillary muscle as an anatomical reference in the image.

The major subsystems of an ECG system are shown at the bottom of the drawing. Electrodes 30 are attached to the skin of the patient at specific locations on the body to acquire ECG signals. Usually the electrodes are disposable conductors with a conductive adhesive gel surface that sticks to the skin. Each conductor has a snap or clip that snaps or clips onto an electrode wire of the ECG system. A typical ECG system will have twelve leads (ten electrodes), which may be expanded with additional leads on the back of the patient for up to sixteen leads. Extended lead sets with up to eighteen leads may be used. In addition, fewer leads such as 3-lead (EASI and other), 5-, and 8-lead sets can also be used to derive 12 leads, but with reduced accuracy The acquired ECG signals, which are on the order of millivolts, are preconditioned by an ECG acquisition module 32 which performs processing such as amplification, filtering and digitizing of the ECG signals. The electrode signals are coupled to an ECG analysis module 36, generally by means of an electrical isolation arrangement 34 that protects the patient from shock hazards and also protects the ECG system when the patient is undergoing defibrillation, for instance. Optical isolators are generally used for electrical isolation. The ECG analysis module combines the signals from the electrodes in various ways to form the desired lead signals, and performs other functions such as signal averaging, heart rate identification, and identifies signal characteristics such as the QRS complex, the P-wave, T-wave, and other characteristics such as elevation seen in the S-T interval. The processed ECG information is then displayed on an image display or printed in an ECG report by an output device 38.

In accordance with the principles of the present invention, the ultrasound images and the ECG lead data are coupled to a combined ultrasound image and ECG display system. In FIG. 1 the ultrasound and ECG information is coupled to an ECG data and ultrasound image data storage device 42. In a typical arrangement the ultrasound system is a stand-alone ultrasound system and the ECG system is a stand-alone cardiograph. Data from the two systems may be directly coupled to the ECG data and ultrasound image data storage device 42, or it may be coupled to the device 42 over a network, or may be ported into the device 42 on one or a plurality of storage media devices. The ECG data and ultrasound image data is then processed for common display by an ECG and ultrasound display processor 40. The merged data is then displayed on an image display 46. A control panel 44 is operated by a user to control the processing and display of the merged data. In a typical implementation, the storage device 42, the processor 40, the control panel 44 and the display 46 are a workstation or a separate computer system.

FIG. 2 illustrates a sequence of operations for acquiring and displaying ultrasound images and ECG lead data in a common display. In step 50 the ultrasound and ECG display system acquires one or more ultrasound images of a desired heart view. The desired view of the heart can be a long or short axis view, a parasternal or apical view, and can be a two- three- or four-chamber view, for example. Next, or concurrently, the ultrasound and ECG display system acquires an ECG lead dataset at step 52. The display system may display all of the twelve ECG lead signals with the ultrasound images, but preferably the display system displays ECG lead signals with ultrasound images that correspond to the view of those ultrasound images. The system may be pre-programmed with certain ECG leads that correspond with specific ultrasound image views, and such programming may be factory-installed and fixed. Preferably, the ECG leads selected for the different ultrasound views are not fixed, but can be varied by the user. In that case, and if the user has a specific set of lead signals to be displayed with a given ultrasound view, the user will select the ECG leads to display with a specific desired view in step 54. At step 56 the display system displays an ultrasound image or loop and its corresponding ECG lead traces on the display 46. The system may also generate an ECG/ultrasound report in step 60 and store or print or transmit the report to another user such as a referring physician in step 62.

FIGS. 3-6 illustrate ultrasound and ECG displays produced by an implementation of the display system of the present invention. In the screen display of FIG. 3, an apical 2-chamber ultrasound view of the heart is shown in the upper display area 72 of the screen. In this example border tracing has been performed to delineate the endocardium and the epicardium of the myocardium in the ultrasound heart image. A border tracing can be drawn on the myocardium of each image of an ultrasound image sequence, then the sequence replayed as a live loop to enable the clinician to view the motion, distortion, and other characteristics of the myocardium as the heart moves. The illustrated border tracing is segmented so that the clinician can refer to a specific segment in a report if an abnormality in a region of the myocardium is diagnosed. If a region of the heart has suffered an infarction, for example, the clinician may diagnose an akinetic condition at a certain segment and so indicate on the diagnostic report. Below the ultrasound image is a display area 74 for ECG traces which correspond to the ultrasound image in display area 72. In this example the 2-chamber view is displaying anterior and inferior segments of the myocardium of the left ventricle and left atrium. The ECG leads which anatomically correspond to this view are anterior leads V3 and V4 and inferior leads II and III or aVF. In this example the traces of leads II, III, V3, and V4 are shown in the lower display area 74.

FIG. 4 shows a 4-chamber apical ultrasound view of the heart in ultrasound display area 76 of the display screen. In this example the myocardium of the left ventricle has been traced and the tracing filled in with colors indicating perfusion by means of contrast agent filling, as explained in U.S. Pat. No. 6,692,438 (Skyba et al.) In this apical 4-chamber view septal and lateral segments of the myocardium of the left ventricle and left atrium are seen, and the ECG leads which anatomically correspond to this view are leads V1 and V2 for the septal segments and leads V5 and V6 for the lateral segments. These lead signal traces are shown in the display area 78 below the ultrasound image.

FIG. 5 shows a display screen with a short axis, mid-cavity view of the heart as the ultrasound image in display area 82. Again, the border of the myocardium has been traced and segmented over the heart myocardium. Since the short axis view shows a complete myocardial path around the heart, anterior, lateral, inferior and septal segments of the myocardium are seen in the ultrasound image. There are a number of ECG lead which anatomically correspond to this view and its segments, including leads aVR, V1 and V2 for the anteroseptal segment, leads aVL, I, V5 and V6 for the anterolateral segment, leads aVF, III, V1 and V2 for the inferoseptal segment, and leads II, aVF, V5 and V6 for the inferolateral segment. In this example leads corresponding to the septal region of the myocardium are displayed in the ECG lead display area 84, which are the aVR, V1, aVR, and III lead signals.

FIG. 6 shows the apical 4-chamber view of the heart of FIG. 4 in the ultrasound display area 92, but in this example the user has selected a different set of leads for concurrent display with this view. As seen in ECG lead display area 94, the user has selected leads V1, V2, V3, and V4 for display with this ultrasound view. At the right side of the ECG lead display area are three columns of ECG lead information. The middle column 98 shows all of the ECG leads of the lead set used for the study. In the left column 96 the user has entered “Xs” next to the leads which are to be displayed in the display area 94. As this example illustrates, the user has selected leads V1, V2, V3 and V4 for viewing. Since the display area can display four lead traces at the illustrated level of resolution, the user can place Xs next to any four leads, and the traces for the four selected ECG leads are shown in the display area 94. The column 90 to the right of the ECG lead column 98 is annotated with the value of ST elevation detected at each lead. In this example the negative values indicate that ST depression has been detected at leads V1, V2, and V3, and so the user has chosen to display the traces for leads V1-V4. The user can save the lead selections corresponding to particular views, such as V1-V4 for the apical 4-chamber view of FIG. 6, and can recall the selections and/or alter them by relocating the Xs in column 96 of the display.

A bullet scorecard is commonly used in ultrasound to record measurements taken at specific segments of the myocardium which correspond to specific segments of the scorecard. In general, a bullet scorecard is an LV segmental display. Ultrasound measurements which are recorded on a bullet scorecard include wall motion values, strain rate values, and perfusion values. The values may be shown quantitatively, but a qualitative bullseye chart is often used to quickly draw the attention of the clinician to a specific heart region. For example, the bullet scorecard 100 in FIG. 7 has been filled in with a green color where wall motion or myocardial perfusion is normal, and has been filled in with red (the darker shade) where abnormal wall motion or myocardial perfusion has been detected. In this example, the attention of the clinician is immediately drawn to the anterior side of the heart anatomy where the abnormality is indicated.

In accordance with the principles of the present invention, a bullseye chart has segments filled in with ECG data corresponding to the anatomical regions of the segments of the chart. The segments of a bullseye chart have been numbered in correspondence with the anatomy of the heart in a standardized pattern as shown in FIG. 8a. Myocardial segments of a basal short axis ultrasound view 102, near the mitral valve plane, are numbered 1 through 6 as shown at the left side of FIG. 8a. The smaller circle 104 represents the segments of a mid-cavity short axis view, with the segments numbered 7 through 12. The lower apical level short axis view 106 has four segments numbered 13 through 16. Each of these three ultrasound image plane circles is oriented to the anterior side of the heart at the top, to the inferior side of the heart at the bottom, to the septal wall to the left and to the lateral wall of the heart at the right. A final segment 17 may be added for the apex of the heart as shown at 108. These circles are displayed concentrically as an ECG bullseye chart 110 as shown in FIG. 8b. The concentric bullseye is three dimensional in nature, as it is anatomically oriented around the chart to the four sides of the heart, and from the outer diameter to the center in accordance with different levels of the heart.

In accordance with a further aspect of the present invention, the bullseye chart is produced with indications of ECG ST elevation values, thus providing an anatomical guide to the location of a possible infarction. The user can consider the ECG bullseye chart alone, or compare it with a bullet scorecard filled in with ultrasonically-derived values for concurrence as to the location, extent, or severity of a heart abnormality. Preferably an ultrasound bullet scorecard and the ECG bullseye chart are displayed side-by-side on the same screen so the user can see the correlation of the results of the two different examinations. Examples of ECG bullseyes illustrating different locations of possible infarction by ST elevation/depression are shown in FIG. 9. FIG. 9a illustrates a condition where abnormal ST elevation values on leads V1 and V2 are mapped to segments 2 and 8 as indicated by the shaded area. Elevated or depressed ST values in these locations are indicative of an affliction in the septal region of the heart (cf. heart locations surrounding ultrasound bullseye of FIG. 7). Since the shaded area is toward the outer periphery of the chart 112, the abnormality is toward the basal heart region. In FIG. 9b the shading of segments 1, 7, and 13 of ECG bullseye chart 114 are mapped from ST elevation on lead aVL and sometimes leads I and/or V2-V3. This abnormality mapping indicates a problem in the mid-anterior region of the heart. In FIG. 9c ST elevation in chart 116 is indicated at segments 13 and 14 and surrounding segments from leads V1-V2 to V3-V6, extending over the apical segment 17. This is indicative of infarction in the apical-anterior region of the heart. FIG. 9d shows an ST bullseye chart 118 which indicates extensive anterior infarction from ST elevation in leads V1-V2 to V4-V6, aVL and sometimes I, mapped to the segments in the upper left half of the bullseye. The bullseye chart 120 of FIG. 9e indicates a lateral mid-cavity condition by the shading of segments 5, 6, 11, and 12 from elevated values on leads V1-V2, I, aVL and V6. The chart 122 of FIG. 9f indicates an inferior condition by the shading of segments 3-4, 9-10 and 15 from elevated ST values on leads II, III and aVF.

In general, the association of abnormal ECG signals to infarcted locations of the heart is as follows:

TABLE 1
Ultrasound View: Apical 4-chamber

| Location | Leads |
|---|---|
| Septal | V1, V2 |
| Lateral | V5, V6 |

TABLE 2
Ultrasound View: Apical 2-chamber

| Location | Leads |
|---|---|
| Anterior | V3, V4 |
| Inferior | II, III, aVF |

TABLE 3
Ultrasound View: Short axis mid-cavity

| Location | Leads |
|---|---|
| Anteroseptal | aVR, V1, V2 |
| Anterolateral | aVL, I, V5, V6 |
| Inferoseptal | aVF, III, V1, V2 |
| Inferolateral | II, aVF, V5, V6 |

It is understood that the above tables are general in nature and that specific physicians may have differing views on the association of specific ECG leads with specific heart regions. Lead placement on the chest can affect the location assignment. Furthermore, new research may find different associations to be more relevant to specific disease conditions.

The values of specific leads can be shown on the ECG bullseye for specific disease conditions being diagnosed. For example, when the clinician is diagnosing hypertrophy, a thickening of the LV wall, the bullseye chart can be scored with the R wave amplitude of lead V5 and the S wave amplitude of lead V1. Amplitudes above certain thresholds, which are age and gender specific, will indicate possible LV wall thickening. Another example is diagnosing atrial enlargement. For left atrial enlargement the P wave amplitudes of leads V1 and V2 will be shown on the bullseye chart. For right atrial enlargement the negative P wave amplitudes of leads aVL and aVR are used. For lead sets with a large number of leads, e.g., a sixteen-lead set, certain leads will view specific heart anatomy from opposite sides of the body and will exhibit corresponding waveforms of opposite polarity. Those skilled in the art will recognized that the values of corresponding leads can be substituted for their opposing leads with due consideration of the difference in polarity.

Other examples of use of the bullseye chart for a particular diagnosis include right ventricle thickening, in which the clinician is diagnosing possible enlargement of the right ventricle. For this diagnosis the values of the R wave amplitude on lead V1 and the S wave amplitude on lead V6 are shown on the bullseye chart. When diagnosing conduction abnormalities for possible cardiac resynchronization therapy, the clinician is looking for indications of left and right bundle branch block. Left bundle branch block is examined by considering the value of left axis shift of the frontal plane vector of the QRS complex for a QRS duration in excess of 120 msec. For right bundle branch block the clinician is examining the right axis shift of the QRS vector.

An implementation of an ECG bullseye chart can be automated, for example, by a processor which fills in segments of the ECG bullseye with characters or colors from the ST elevation values given for each ECG lead in column 90 of FIG. 6. The map of ECG leads to specific segments of the bullseye chart can be adjusted by the user to reflect the user's judgment on the correct association of ECG leads to bullseye segments. Other variations will readily occur to those skilled in the art. For example, segments with normal ST elevation values can be colored green, segments with elevated ST values (e.g., greater than one millivolt) can be colored red, and segments with depressed ST values (e.g., less than minus one millivolt)can be colored blue, thereby giving the user a sense of problem areas and the data indicating those abnormalities.

What is claimed is:

1. A diagnostic system for ultrasound image and ECG lead signal data comprising:
a source of ultrasound images of a heart acquired from one or more viewing perspectives;
a source of ECG lead signal data;
a display processor, responsive to the ultrasound images and the ECG lead signal data which is adapted to produce a common display of an ultrasound image and ECG lead signal data corresponding to the viewing perspective of the ultrasound image; and
a display device, coupled to the display processor, for displaying the common ultrasound and ECG display.

2. The diagnostic system of claim 1, further comprising a data storage device, responsive to ultrasound image data and ECG lead signal data, and coupled to the display processor.

3. The diagnostic system of claim 1 wherein the ECG lead signal data further comprises traces of ECG waveforms.

4. The diagnostic system of claim 3, wherein the source of ECG lead signal data further comprises signals of at least twelve ECG leads, and wherein the display processor processes signals of fewer than twelve leads for display on the common display.

5. The diagnostic system of claim 4, wherein the display processor is further adapted to process signals of up to four leads for display on the common display.

6. The diagnostic system of claim 4, further comprising a selection of more than four ECG leads from which a user can select a subset of the selection for common display with the ultrasound image.

7. The diagnostic system of claim 1, wherein an ultrasound image is obtained with a viewing perspective showing the motion or tissue distortion of a region of the heart,
wherein the ECG lead signal data corresponding to the viewing perspective comprises signals of ECG leads which are in closer proximity to the region of the heart than other nondisplayed ECG lead signal data.

8. The diagnostic system of claim 1, wherein the ultrasound image viewing perspective comprises one of a 4-chamber view, a 2-chamber view or a short axis view, and wherein the ECG lead signal data displayed comprises the data of one or more ECG leads which are in physical proximity to the tissue seen in the ultrasound image.

9. The diagnostic system of claim 8, wherein the ECG lead signal data further comprises ECG signals received from a lateral side, septal side, anterior side, or inferior side of the heart.

10. The diagnostic system of claim 1, wherein the display processor is further adapted to display in the common display a list of all of the leads represented by the ECG lead signal data.

11. The diagnostic system of claim 10, further comprising a user-operated control which produces a selection signal; and
wherein the display processor is further responsive to the selection signal to display one or more of the leads on the lead list as selected for display of a trace in the common display.

12. The diagnostic system of claim 11, wherein the display processor is further responsive to the selection signal to indicate on the common display that one or more of the leads of the lead list have been selected for display in the common display.

13. The diagnostic system of claim 10, wherein the source of ECG lead signal data further comprises ST elevation data for a plurality of ECG leads;
wherein the display processor is responsive to the ST elevation data for displaying ST elevation data for a plurality of the leads on the lead list.

14. The diagnostic system of claim 13, wherein the display processor is responsive to the ST elevation data for displaying ST elevation data for all of the leads on the lead list.

15. The diagnostic system of claim 1, wherein the display processor is adapted to produce a common display of at least one of the following:
signals of leads V1 and V2 in correspondence with an ultrasound image of a septal view of the heart;
signals of leads V5 and V6 in correspondence with an ultrasound image of a lateral view of the heart;
signals of leads V3 and V4 in correspondence with an ultrasound image of an anterior view of the heart; and
signals of leads II, III, or aVF in correspondence with an ultrasound image of an inferior view of the heart;
